(19) United States
(12) Patent Application Publication
(10) Pub. No.: US 2026/0354851 A1
(43) Pub. Date: Nov. 12, 2026

(54) SHOCK WAVE CATHETER WITH SHIELDED EMITTERS

(71) Applicant: Shockwave Medical, Inc., Santa Clara, CA (US)

(72) Inventors: Todd Weston JENKINS, San Jose, CA (US); Rainier BETELIA, San Jose, CA (US); Jason B. FRANCO, Milpitas, CA (US); Nancy P. NGUYEN, San Jose, CA (US)

(73) Assignee: Shockwave Medical, Inc., Santa Clara, CA (US)

(21) Appl. No.: 19/204,227

(22) Filed: May 9, 2025

(51) Int. Cl. A61B 17/22

(52) U.S. Cl. CPC A61B 17/22022; A61B 2017/22025

(57) ABSTRACT

A catheter for treating a lesion in a body lumen includes an elongate member; an enclosure surrounding a distal region of the elongate member; a shock wave emitter located within the enclosure; and a shield positioned between the shock wave emitter and the enclosure such that a shock wave emitted by the shock wave emitter passes through the shield before passing through the enclosure.

### FIELD OF THE DISCLOSURE

The present disclosure relates generally to the field of medical devices and methods, and more specifically to acoustic energy generating assemblies for inclusion in catheter devices used for treating lesions in a body lumen, such as calcified lesions and occlusions in vasculature, calcific valve stenosis, and kidney stones in the urinary system.

### BACKGROUND

Calcified lesions in body lumens can negatively impact patient health. For example, when calcium builds up in the walls of the coronary arteries, the calcification can restrict blood flow to the heart muscle, which can eventually lead to a heart attack. Catheter devices are one type of device that can be used to treat calcified lesions in a body lumen. When treating lesions with a catheter device, it is important to minimize damage to surrounding soft tissues while still breaking up the lesion as much as possible.

A wide variety of catheters have been developed for treating calcified lesions stenotic blood vessels that are narrowed by the progressive growth and accumulation of plaque, a condition also known as atherosclerosis. A particular focus is to treat calcified lesions in the vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature.

However, traditional balloon angioplasty therapies may not work with calcified tissue because the calcium in the atherosclerotic plaque hardens the lesion, resisting the mechanical force of balloon expansion. The resistance can result in more procedural complication and vessel damage because the high-pressure balloons preferentially expand away from the hard calcified tissue. The predisposition of the ballon to expand in a direction of lower resistance increases the risk of major dissection or perforation of the vessel, often at the interface between healthy tissue and calcified tissue (i.e., where the balloon encounters soft tissue), or in the case of an eccentric calcified lesion, in the direction within the blood vessel opposite of the calcified region of the lesion. Moreover, in the case of nodular calcium, expansion of a standard angioplasty balloon can lead to pushing the node of calcified material in a manner that may puncture the vessel.

More recently, the technique and treatment of intravascular lithotripsy (IVL) has been developed, which is an interventional procedure to modify calcified plaque in diseased arteries. The mechanism of plaque modification is through use of a catheter having one or more ultrasonic short pressure pulses (commonly referred to as “shock waves”) generating sources located within a liquid that can generate ultrasonic shock waves that modify the calcified plaque. IVL devices vary in design with respect to the energy source used to generate the acoustic shock waves, with two exemplary energy sources being electrohydraulic generation and laser generation.

Another approach to dealing with calcified stenotic plaque is to cut away at a calcified lesion, by using a cutting or scoring balloon (e.g., an angioplasty balloon with blade-like structures on the exterior), or by using an atherectomy device which is typically a motor-driven rotating blade that is pushed onto and cuts through an occlusion. The IVL process can also be considered different from standard atherectomy procedures in that it cracks calcium but does not liberate the cracked calcium from the tissue. Hence, generally speaking, IVL should not require aspiration nor embolic protection. Moreover, the movement or rotation of an atherectomy catheter blades generates heat and related thermal injury from operation of the atherectomy device leads to an increased risk of blood clotting. Naturally, the action of a moving blade within the vasculature also significantly increases the potential for a large dissection and perforation of the blood vessel. In further contrast with cutting techniques, due to the compliance of a normal blood vessel and non-calcified plaque, the shock waves produced by IVL do not modify the normal vessel tissue or non-calcified plaque. Accordingly, IVL does not carry the same degree of risk of embolism, perforation, dissection, or other damage to vasculature as atherectomy procedures or angioplasty procedures using cutting or scoring balloons.

More specifically, catheters to deliver IVL therapy have been developed that include pairs of electrodes for electrohydraulically generating shock waves inside an angioplasty balloon. Shock wave devices can be particularly effective for treating calcified plaque lesions because the pressure from the shock waves can crack and disrupt lesions near the angioplasty balloon without harming the surrounding tissue. In these devices, the catheter is advanced over a guidewire, through a lumen, or directly delivered percutaneously through a patient's vasculature until it is positioned proximal to and/or aligned with a calcified plaque lesion in a body lumen. The balloon is then inflated with conductive fluid (e.g., using a relatively low pressure of 2-4 atm) so that the balloon expands to contact or be in apposition with the lesion but not to a degree that substantively displaces the lesion. Voltage pulses can then be applied across the electrodes of electrode pairs to produce ultrasonic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can include electrodes disposed within a closed volume other than an angioplasty balloon, such as a cap, balloons of variable compliancy, or other type of enclosure.

Enclosures, such as angioplasty balloons, are typically configured to minimize the amount of shock wave energy dissipation. For example, the walls of the enclosures may be sufficiently thin and pliable as to appear acoustically transparent to the shock waves. Thin and pliable enclosure may provide the additional benefit of increasing flexibility of the catheter and reducing its crossing profile. However, the walls of the enclosures must be strong enough to handle the inflation pressures used to fill the enclosures with fluid. Defects or damage to an enclosure may lead to rupture when under pressure. A ruptured enclosure may be incapable of inflating, preventing fluid from surrounding shock wave emitters and, thereby, preventing the generation of shock waves.

### SUMMARY

According to an aspect, a catheter includes a shield disposed between a shock wave emitter and an enclosure to protect the enclosure from heat and/or debris generated by the shock wave emitter during use. The shield may be a tubular body with at least one open end that enables fluid within the enclosure to flow within and around the shield. The shield may be free-floating within the enclosure. Optionally, the shield is spaced apart from the shock wave emitter by at least one spacer.

According to an aspects, a catheter for treating a lesion in a body lumen includes: an elongate member; an enclosure surrounding a distal region of the elongate member; a shock wave emitter located within the enclosure; and a shield positioned between the shock wave emitter and the enclosure such that a shock wave emitted by the shock wave emitter passes through the shield before passing through the enclosure.

The shield may be open at at least one end. The shield may be free-floating within the enclosure. The catheter may include a spacer that maintains a minimum spacing between the shock wave emitter and the shield. The spacer may be configured to compress. At least a portion of the spacer may be moveable relative to the elongate member to enable the spacer to compress. The catheter may include a retainer for retaining the spacer in position relative to the elongate member.

The shield may be tubular in shape. The shield may be configured as a strip. The shock wave emitter may include an electrode pair that includes a spark gap and the shield may be located between the spark gap and the enclosure.

The shield may include a thermoplastic polyurethane or a polyether block amide. The shield may include a material having a specific gravity of 1.0-1.6. The shield may include a material having a Shore A hardness less than 100. The shield may include a radiopaque material.

According to an aspect, a method of treating a lesion in a body lumen includes navigating a shock wave catheter through the body lumen; positioning the shock wave catheter such that a shock wave emitter of the shock wave catheter is proximate to the lesion, wherein a shield of the shock wave catheter is located between the shock wave emitter and the enclosure; and operating the shock wave catheter to generate one or more shock waves by the shock wave emitter to treat the lesion.

The method may include filling the enclosure with fluid, wherein filling the enclosure with fluid causes the enclosure and the shield to expand. The shield may be open at at least one end. The shield may be free-floating within the enclosure. The catheter may include a spacer that maintains a minimum spacing between the shock wave emitter and the shield. The spacer may be configured to compress.

### DESCRIPTION OF THE FIGURES

Illustrative aspects of the present disclosure are described in detail below with reference to the following figures. It is intended that the embodiments and figures disclosed herein are to be considered illustrative and exemplary rather than restrictive.

FIG. 1 illustrates a system that includes a shock wave catheter for treating a hardened lesion in a cardiovascular system, according to aspects of the disclosure;

FIGS. 2A and 2B illustrate aspects of a distal portion of a catheter that includes a shield configured as a cylindrical tube, according to aspects of the disclosure;

FIGS. 3A and 3B illustrate an exemplary shock wave catheter in which spacing between a shield and shock wave emitters is maintained by one or more spacers, according to aspects of the disclosure;

FIG. 4A illustrates a catheter that includes an exemplary configuration of a spacer for spacing a shield from one or more shock wave emitters, according to aspects of the disclosure;

FIG. 4B illustrates an exemplary retainer that is configured to maintain a position of the spacer along the elongate member of a catheter, according to aspects of the disclosure;

FIG. 4C illustrates an alternative configuration of a retainer for retaining a spacer on an elongate member, according to aspects of the disclosure;

FIG. 5 illustrates a catheter that includes shield strips that are positioned in front of shock wave emitters, according to aspects of the disclosure; and

FIG. 6 illustrates an example of a computing system, according to aspects of the disclosure.

### DETAILED DESCRIPTION

The following description is presented to enable a person of ordinary skill in the art to make and use the various embodiments and aspects thereof disclosed herein. Descriptions of specific catheters, systems, methods, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

For electrohydraulic generation of ultrasonic short pressure pulses used in IVL, a conductive solution (e.g., saline) can be contained within an enclosure that surrounds electrodes or can be flushed through a tube that surrounds the electrodes. The calcified plaque modification is achieved by creating ultrasonic shock waves within the catheter by an electrical discharge (e.g., a plasma arc) across the electrodes. The energy from this electrical discharge enters the surrounding fluid, generating an acoustic shock wave where the wave itself is ultrasonic (i.e., having a wave frequency of greater than 20,000 Hz), as well as a vapor bubble. In addition, this discharge creates one or more rapidly expanding vapor bubbles that generate secondary shock waves due to the cavitation of the collapsing vapor bubble. These shock waves propagate radially outward and modify calcified plaque within the blood vessels. The shock waves travel deeply and safely through soft arterial tissue because of the acoustic impedance soft tissue, which is similar to water. When the shock waves encounter tissues with a different acoustic impedance, such as intimal calcification of plaque close to the surface or endothelium of a vessel or medical calcification in the smooth muscle layer of a vessel, the leading edge of the shock wave imparts compressive stress on the calcified tissue. Shearing occurs on the lesion as the shock wave passes through the calcification. When the shock wave reaches the distal boundary of the calcification, the shock wave is both transmitted and reflected, inducing tensile stress that pulls the calcification apart. Further compressive stress is applied by the squeezing which occurs when the ultrasonic shock wave entering the calcium propagates faster than the remaining shock wave travelling outside the calcified region of tissue. These forces generated by IVL result in multi-plane and longitudinal fractures of the calcification in the tissue.

For laser generation of acoustic shock waves, a laser pulse is transmitted into and energy from the laser is absorbed by a fluid within the catheter, optionally with a target to act as catalyst for the laser absorption. This absorption process rapidly heats and vaporizes the fluid, thereby generating the rapidly expanding vapor bubble, as well as the acoustic shock waves that propagate outward and modify the calcified plaque. The acoustic shock wave intensity is higher if a fluid is chosen that exhibits strong absorption at the laser wavelength that is employed. These examples of electrohydraulic and laser-based IVL devices are not intended to be a comprehensive list of potential energy sources to create the ultrasonic IVL shock waves.

According to various aspects, a catheter includes at least one shock wave emitter enclosed within an enclosure and a shield disposed between the shock wave emitters and the enclosure to protect the enclosure from debris and/or heat generated by the shock wave emitters when generating shock waves. Shock wave generation may cause material erosion (e.g., of electrode materials) and release a significant amount of heat, which can damage or wear down an enclosure. This phenomenon may be particularly problematic when treating lesions of larger body lumens and structural heart features, such as heart valves, where stronger shock waves may be necessary, because generating stronger shock waves generally requires a higher energy input, which in turn leads to accelerated material erosion and more heat generation. Catheters disclosed herein include shields that block debris from impacting the enclosure and reduce the amount of heat that reaches the enclosure. By protecting the enclosure from debris and/or heat, the shields may increase the lifespan of the enclosure (and, thereby, the catheter), reduce the chance of early failure, and/or enable an enclosure to be made thinner and/or more flexible.

The shield may have a tubular shape and may surround the shock wave emitters. The shield may include at least one open end that allows fluid to flow within and around the shield. Since the shield is not a pressure vessel, the shield is not under tension during use and, therefore, is less susceptible to damage by debris and/or heat than a pressurized enclosure.

The shield may be free-floating within the enclosure. Spacing between the shield and the shock wave emitter may be maintained by at least one spacer. The spacer may be compressible to enable the catheter to have a lower profile, such as for delivery to a treatment site or removal from a treatment site. In some examples, the shield includes a plurality of separate shield portions that extend in front of the shock wave emitters.

As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement. Furthermore, numerical designators such as “first,” “second,” “third,” “fourth,” etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode, or in an arrangement of a distal electrode and proximal electrode along a length of a catheter. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). In some contexts, one or more electrode pairs may also be referred to as an electrode assembly. In the context of the present disclosure, the term “emitter” broadly refers to the region of an electrode assembly where the current transmits across the one or more electrode pairs, generating at least one shock wave. The term “emitter sheath” or “emitter band” (which are used interchangeably) refers to a sheath/band of conductive material that may form one or more electrodes of one or more electrode pairs, thereby forming a location of one or more emitters.

Components of emitters, including electrodes and emitter sheaths/bands, may be formed from a metal, such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or a mixture of such materials.

In some embodiments, an IVL catheter may be an “over-the-wire-type” (OTW) catheter. In an OTW catheter, the guidewire entry point to the catheter is at the distal end of the catheter tip, extends through the full length of the catheter through a lumen (alternatively referred to as a “guidewire lumen” and sometimes is structurally also a “central lumen”), and exits from the proximal end of the catheter via a guidewire port. In other embodiments, an IVL catheter is a “rapid exchange” (Rx) catheter. An Rx design allows just a portion of the catheter to ride over a guidewire. The guidewire entry point to the catheter is at the distal end of the catheter tip, and the extends along a pathway (e.g., a dedicated guidewire lumen) through a portion of the catheter until it reaches an exit port on the side of the catheter. The guidewire exit port is generally located at a position behind (proximal from) the distal tip of the catheter, and moreover the exit port is located along the length of a catheter proximal to a functional region of the catheter (e.g., an expandable member or a tensioned member having shock wave emitters). The location of an Rx catheter guidewire exit port can vary for a given design, generally being positioned from fifteen to thirty-five centimeters (15-35 cm) behind the distal tip, although it is appreciated that in some implementations the exit port can be less than 15 cm or greater than 35 cm behind the distal tip of the catheter. Thus in use, a guidewire is delivered into the anatomy of a patient, the proximal end of the guidewire (outside the patient) is fed into the distal end opening of the catheter, and the catheter is run along the guidewire until it reaches the target tissue at the distal end of the guidewire (inside the patient); the effective difference between an OTW and an Rx catheter is where the guidewire exits the catheter. The selection between an OTW design and an Rx design is driven by factors including (but not limited to): anatomy to be treated (e.g., coronary vasculature vs. peripheral vasculature); the length of guidewire to be used; the trackability, stiffness, torque transmission, and deliverability of the catheter; the profile and cross-section of the catheter, the ability to exchange a wire when the catheter is past a stenosis; positioning of the distal end of a catheter close to the end of a guidewire and further obtaining positional confirmation of the catheter.

For treatment of an occlusion in a blood vessel, the voltage pulse applied by a power source, including any of the power sources described herein (which may also be referred to herein as voltage sources or pulse generators), is typically in the range of from about five hundred to three thousand volts (500 V-3,000 V). In some implementations, such as for treatment of calcification on structural heart anatomy, the voltage pulse applied by the voltage source can be up to about fifteen thousand volts (15,000 V) or higher than fifteen thousand volts (15,000 V). The pulse width of the applied voltage pulses ranges between one microsecond and six microseconds (1-6 μs). The repetition rate or frequency of the applied voltage pulses may be between about 1 Hz and 10 Hz. The total number of pulses applied by the power source to a treatment device (e.g., an IVL catheter) can be, for example, sixty (60) pulses, eighty (80) pulses, one hundred twenty (120) pulses, three hundred (300) pulses, or up to five hundred (500) pulses, or any increment of pulses within this range. Further implementations of power sources can deliver greater than 500 pulses to a treatment device. Alternatively or additionally, in some examples the power source may be configured to deliver a packet of micro-pulses having a sub-frequency between about 10 Hz-10 kHz. The preferred voltage, repetition rate, and number of pulses for any given IVL device or treatment may vary depending on factors such as the size, length, eccentricity, nodularity, or orientation of the lesion, the extent of calcification, the size of the blood vessel, the attributes of the patient, or the stage of treatment. For instance, a physician may start with low energy shock waves and increase the energy as needed during the procedure, or vice versa. The magnitude of the shock waves can be controlled by controlling the voltage, current, duration, and repetition rate of the pulsed voltage from the power source.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs can be found in U.S. Pat. Nos. 8,888,788, 9,433,428, 10,555,744, and 10,709,462, in U.S. Publication No. 2021/0085383, and in U.S. patent application Ser. No. 18/586,299, all of which are incorporated herein by reference in their entireties. Other catheter designs have improved the delivery of shock waves, for instance, by specific electrode construction and configuration thereby directing shock waves in a forward direction to break up tighter and harder-to-cross occlusions in vasculature. Examples of forward-biased or firing-firing catheter designs can be found in U.S. Pat. Nos. 10,966,737, 11,478,261, and 11,596,423, in U.S. Publication Nos. 2023/0107690 and 2023/0165598, and in U.S. patent application Ser. No. 18/524,575 and Ser. No. 18/680,853, all of which are incorporated herein by reference in their entireties. Efforts have also been made to develop catheters to deliver shock waves to structural heart anatomy. Examples of catheter designs for structural heart treatment can be found in U.S. Pat. Nos. 9,044,618 and 10,646,240.

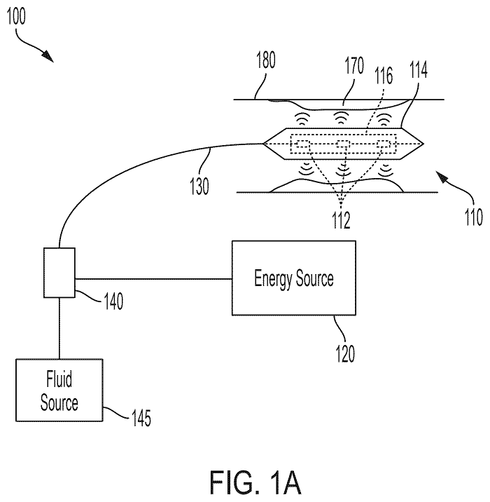

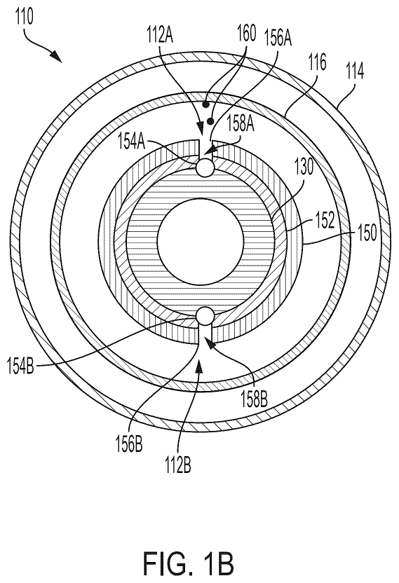

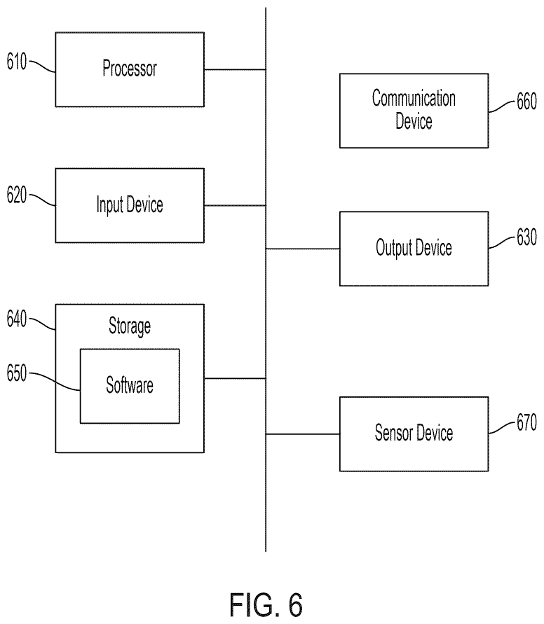

FIG. 1A illustrates a system 100 that includes a shock wave catheter 110 for treating a hardened lesion in a cardiovascular system, according to aspects of the disclosure. The catheter 110 includes one or more shock wave emitters 112 carried by an elongate member 130. The system may include an energy source 120 connected by one or more energy guides extending in or along the elongate member 130 to the one or more shock wave emitters 112 for delivering energy to generate shock waves at the shock wave emitters 112. System 100 may include a control hub 140 for controlling energy delivery and fluid flow from a fluid source 145 to the distal working region of catheter 110. Energy source 120 may include a high voltage pulse generator and a power supply. In some examples where catheter 110 is intended to treat larger body lumens or heart valves, energy source 120 may include a voltage pulse generator capable of generating voltage pulses of, for example, from 6 kV to 20 kV, of from 8 kV to 18 kV, or from 10 kV to 15 kV. Energy source 120 may include or be connected to a computing system, such as the system described below with reference to FIG. 6

Catheter 110 includes, at its distal region, an enclosure 114 that may be fillable with a fluid from the fluid source 145. The enclosure 114 may be an angioplasty balloon that may be inflated at a treatment site to a relatively low pressure (e.g., less than 5 atm) before shock waves are generated at the shock wave emitters 112. The fluid used to fill the enclosure 114 may include saline. Optionally, the fluid includes a contrast agent, such as a fluoroscopic contrast agent. In some examples, the fluid includes a mixture of saline and contrast agent.

FIG. 1B is a cross section of an exemplary configuration of catheter 110 illustrating an exemplary configuration of shock wave emitters 112. In the example of FIG. 1B, the shock wave emitters are configured as electrode pairs that generate shock waves via application of a voltage pulse. Catheter 110 includes a conductive band 150 that is separated from the elongate member 130 by an insulation layer 152. A first conductor 154A extends along a first side of the elongate member 130 (e.g., with a lumen or groove of the elongate member 130), and a second conductor 154B extends along a second side of the elongate member 130. A first hole 156A is provided in the conductive band 150 at the location of the first conductor 154A and a second hole 156B is provided in the conductive band 150 at the location of the second conductor 154B. An end of the first conductor 154A may be positioned adjacent the first hole 156A and an end of the second conductor 154B may be positioned adjacent the second hole 156B. The first conductor 154A and the conductive band 150 are spaced apart by a first spark gap 158A, and the second conductor 154B and the conductive band 150 are spaced apart by a second spark gap 158B. The first conductor 154A and the portion of the conductive band 150 at the first hole 156A are a first pair of electrodes that form a first shock wave emitter 112A. The second conductor 154B and the portion of the conductive band 150 at the second hole 156B are a second pair of electrodes that form a second shock wave emitter 112B. A voltage pulse applied across the first conductor 154A and the second conductor 154B (e.g., a voltage pulse generated by energy source 120) results in current flowing between the first conductor 154A and the conductive band 150 (across the first spark gap 158A), through the conductive band 150, and between the conductive band 150 and the second conductor 154B (across the second spark gap 158B). Current flows across the first spark gap 158A as a spark, which results in a shock wave being emitted from the first shock wave emitter 112A. Similarly, current flows across the second spark gap 158B as a spark, which results in a shock wave being emitted from the second shock wave emitter 112B.

In other implementations, instead of electrode pairs, a shock wave may be generated when an energetic light pulse (e.g., a laser light pulse) delivered by an optical fiber is absorbed by a target (e.g., water molecules) in the enclosure. In such examples, a shock wave emitter can include a distal end (or other light emitting region) of an optical fiber.

Shock wave generation may cause material erosion (e.g., of electrode materials) and release a large amount of heat, which can damage or wear down an enclosure. This phenomenon may be particularly problematic when treating lesions of larger body lumens and structural heart features, such as heart valves, where stronger shock waves may be necessary, because generating stronger shock waves generally requires a higher energy input, which in turn leads to accelerated material erosion and more heat generation. Catheter 110 includes a shield 116 located inside of the enclosure 114 between the shock wave emitters 112 and the enclosure 114. Shield 116 may function as a physical barrier between shock wave emitters 112 and enclosure 114 to protect enclosure 114 from debris generated by erosion of shock wave emitters 112 (e.g., electrode material) and heat generated during shock wave generation. For example, with reference to FIG. 1B, debris 160 that is generated by the first shock wave emitter 112A is prevented by the shield 116 from impacting the enclosure 114.

Shield 116 may be substantially acoustically transparent to the shock waves generated by shock wave emitters 112. The protection of the enclosure 114 provided by shield 116 may increase the useful lifespan of the catheter 110 relative to a similar catheter that does not include the shield 116. The protection provided by the shield 116 can be either or both of mechanical protection against debris ejected from and thermal protection against heat generated by shock wave emitters 112. Additionally, or alternatively, the inclusion of shield 116 may enable the enclosure to be made of a thinner and/or more flexible material, which may increase the flexibility of the catheter 110 and/or reduce its crossing profile.

With reference to FIG. 1A, catheter 110 may be used for treating a lesion 170 in a body lumen 180 by navigating the catheter 110 through the body lumen 180 and positioning the catheter 110 such that at least one shock wave emitter 112 is proximate to the lesion 170. The catheter 110 may be operated to generate one or more shock waves by the shock wave emitter 112 to treat the lesion 170. For example, the energy source 120 may be operated to apply one or more voltage pulses to the shock wave emitter 112.

Prior to generating the shock waves, the enclosure 114 may be filled with a fluid, such as a conductive fluid (e.g., saline), provided by fluid source 145. Prior to filling the enclosure 114 with the fluid, the enclosure 114 may be in a collapsed state, and filling the enclosure 114 with the fluid may cause the enclosure 114 to expand into an expanded state. When the enclosure 114 is in its collapsed state, the shield 116 may be in a compressed state in which the collapsed enclosure 114 compresses the shield 116 into a smaller profile. As such, expanding the enclosure 114 via filling may enable the shield 116 to expand into a uncompressed state. For example, the shield 116 may be formed of a resilient, but flexible, material such that the shield 116 may be compressed into a smaller profile but will return to a nominal (e.g., tubular) profile upon removal of compression forces (e.g., from the collapsed enclosure).

Optionally, after treatment of the lesion with shock waves, the enclosure 114 may be returned to a collapsed state by withdrawing the fluid, which may apply a vacuum pressure to the enclosure 114. Collapsing of the enclosure 114 may cause compression of the shield 116 into a smaller profile. With the enclosure 114 in a collapsed state and the shield 116 in a compressed state, the catheter 110 may be repositioned with the body lumen. For example, the catheter 110 may be moved to another portion of the lesion, moved to a different lesion, and/or withdrawn from the body lumen.

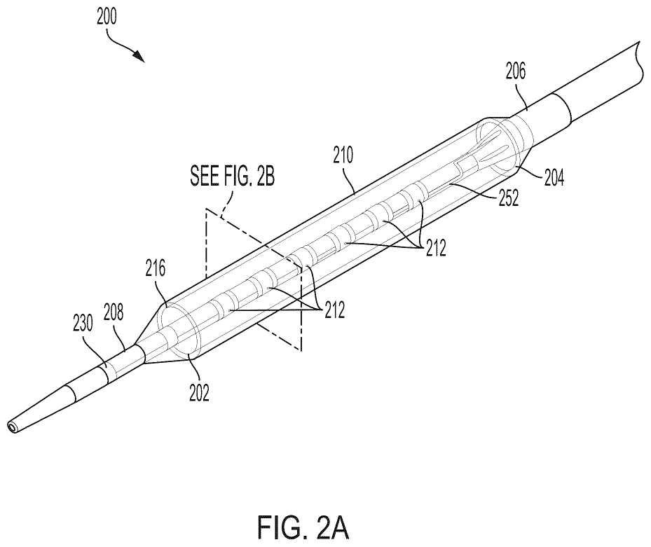

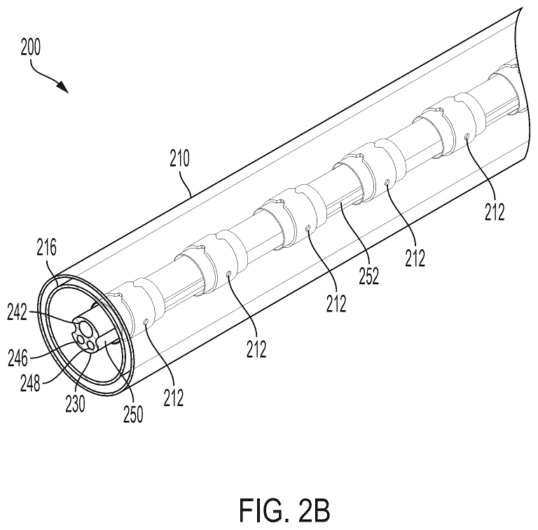

FIGS. 2A and 2B illustrate aspects of a distal portion of a catheter 200 in which a shield 216 is configured as a cylindrical tube. Catheter 200 can be used for catheter 110 of FIGS. 1A and 1B. FIG. 2B is a cross-section of the catheter 200 taken with respect to plane A of FIG. 2A. Catheter 200 includes a plurality of shock wave emitters 212 carried by an elongate member 230. Enclosure 210 is attached to the elongate member 230 and encloses the shock wave emitters 212. Shield 216 is positioned with enclosure 210 and encircles the shock wave emitter 212 such that shield 216 is located between shock wave emitters 212 and enclosure 210.

Shield 216 may help to prolong the life of enclosure 210 (and, thus, prevent catheter failure) by acting as a physical barrier between the shock wave emitters 212 and enclosure 210. Shield 216 may also help to prolong the durability of enclosure 210 by acting as a thermal barrier, reducing the amount of heat generated by the shock wave emitters 212 that reaches enclosure 210. As shown in FIGS. 2A-2B, shield 216 may have a tubular shape and has a length sufficient for the shield 216 to cover all of the shock wave emitters 212. Shield 216 may include an open distal end 202 and/or an open proximal end 204 that allows fluid and/or any generated gas bubbles to flow freely within shield 216 and around shield 216. Because of the open distal and/or proximal ends, shield 216 is not pressurized and, thus, is not under tensile stress. Because shield 216 is not in tension, impact to shield 216 by debris from shock wave emitter 212 causes less damage than impact to enclosure 210 when pressurized. As such, the inclusion of shield 216 is better at reducing the chances of rupture of enclosure 210 than a commensurate increase in wall thickness of enclosure 210.

Shield 216 may be free-floating (i.e., not attached to other elements of catheter 200), which may simplify inclusion of shield 216 during assembly of catheter 200. A catheter having a free-floating shield may also be more navigable and flexible than a comparable catheter having a shield that is not free-floating. A free-floating shield that can be compressed when enclosure 210 is not filled may also allow for a more compact crossing profile during catheter delivery.

Enclosure 210 may be sealed at its proximal end 206 and distal end 208 to elongate member 230. The proximal end 206 of enclosure 210 may be located proximally of shield 216. The distal end 208 of enclosure 210 may be located distally of shield 216.

Because the shield 216 is not attached to the enclosure 210, the shield 216 may shift in position longitudinally and/or laterally relative to the elongate member 230. However, shield 216 may have a diameter that is sufficiently large such that when the shield 216 shifts laterally a maximum amount (i.e., the shield 216 contacts the enclosure 210), the shield 216 cannot contact the shock wave emitters 212. In other words, the shield 216 may be configured such that if one side of the shield 216 contacts enclosure 210, the opposite side of the shield 216 is spaced from the shock wave emitters 212. Additionally, or alternatively, the length of the shield 216 may be such that when the shield 216 shifts longitudinally a maximum amount (e.g., an end of the shield 216 contacts an end portion of the enclosure 210), all of the shock wave emitters 212 remain covered by the shield 216. Further, the dimensions of shield 216 may allow the shield 216 to seat itself alongside the inner surface of enclosure 210 without need for manipulation to that location by an operator.

Catheter 200 may include, as part of elongate member 230, lumina 242, 244, 246. Other examples may include fewer or more lumina extending within elongate member 230. Lumina 242, 244, 246 may be provided to accommodate a guidewire, to allow fluid exchange from enclosure 210, and/or to accommodate one or more energy guides. Elongate member 230 may include one or more grooves 250 extending along its surface to accommodate one or more energy guides, such as one or more wires 252.

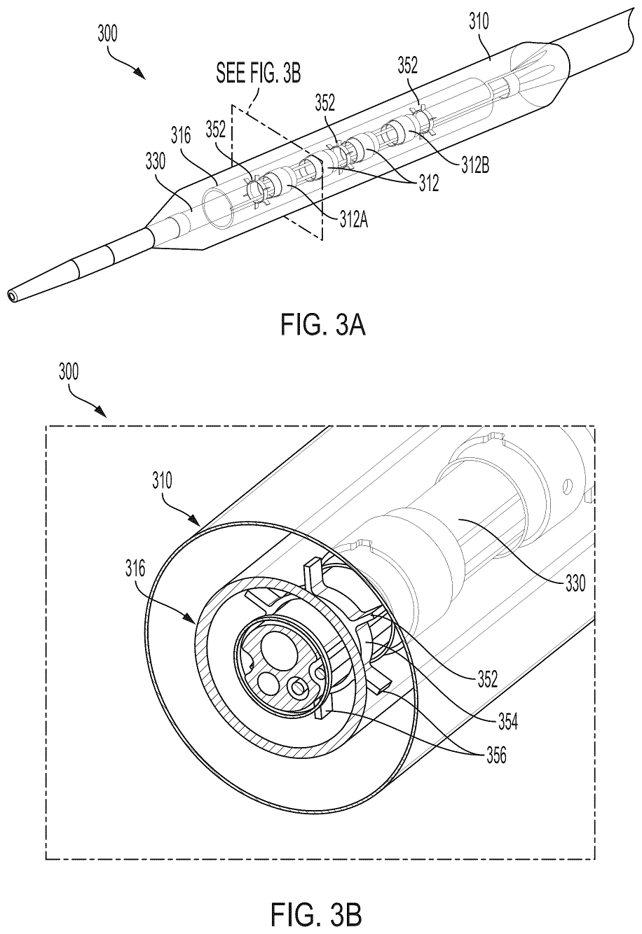

Spacing between a shield and shock wave emitters may be maintained by including one or more spacers. FIGS. 3A and 3B illustrate an exemplary shock wave catheter 300 having an enclosure 310 in which spacing between a shield 316 and shock wave emitters 312 is maintained by one or more spacers 352. FIG. 3B is a cross-section of the catheter 300 taken with respect to plane B of FIG. 3A. Catheter 300 may be used for catheter 110 of FIG. 1. Spacers 352 may be configured to ensure flow of fluid and gas bubbles proximate the shock wave emitters 312. Spacers 352 may be configured to ensure that shield 316 is at least 0.5 mm away from shock wave emitters 312. Spacers 352 may be configured to ensure that shield 316 is located in a range from 0.5 mm to 3.0 mm away from shock wave emitters 312. In some examples, spacers 352 are formed of a shape memory material, such as nitinol. The illustrated example includes three spacer 352—one located between a center two shock wave emitters 312, one located distally of a distal-most shock wave emitter 312A, and one located proximally of a proximal-most shock wave emitter 312B. However, this arrangement is merely exemplary and any number and arrangement of spacers 352 may be used.

Spacers 352 may include an annular body 354 that has an inner diameter that is large enough to fit around elongate member 330. Optionally, spacers 352 are fixed in place by an adhesive. In some examples, spacers 352 are kept in place by a frictional fit to the elongate member 330. In other examples, spacers 352 are free to move along the elongate member 330.

The spacers 352 may include projections 356 that project from the annular body 354. The projections 356 may be evenly spaced about a circumference of the annular body 354. The projections 356 project from the annular body 354 by an amount sufficient to maintain a desired spacing between the shock wave emitters 312 and the shield 316. The projections 356 may be configured to hold the shield 316 in place. For example, the shield 316 may be attached to the projections 356. Alternatively, the projections 356 may be configured to leave an amount of clearance between ends of the projections 356 and the shield 316 so that shield 316 is free-floating. In implementations where the spacers 352 and/or the projections 356 are made from a shape memory material, such components can have a collapsed or reduced-profile configuration when the catheter 300 is being delivered to a target anatomy, and subsequently such components can have a restored or expanded-profile configuration when positioned at the target anatomy to effectuate spacing functionality as described herein.

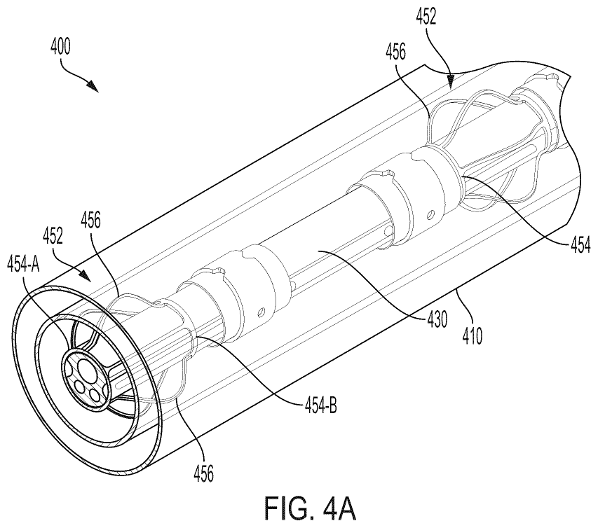

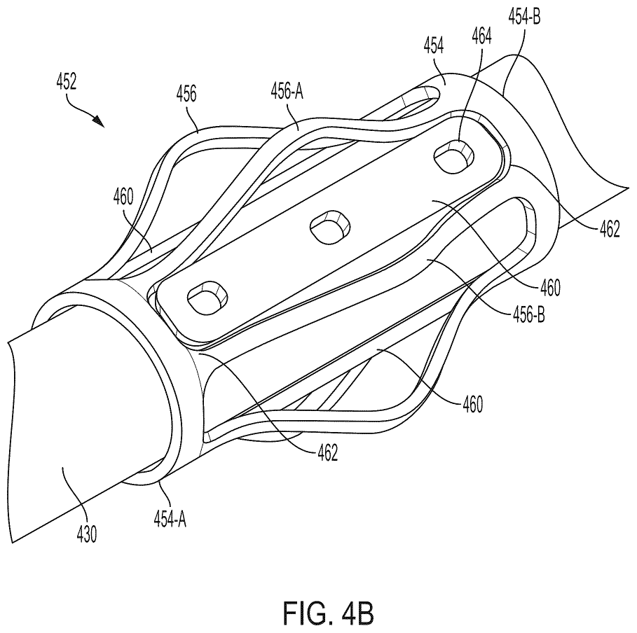

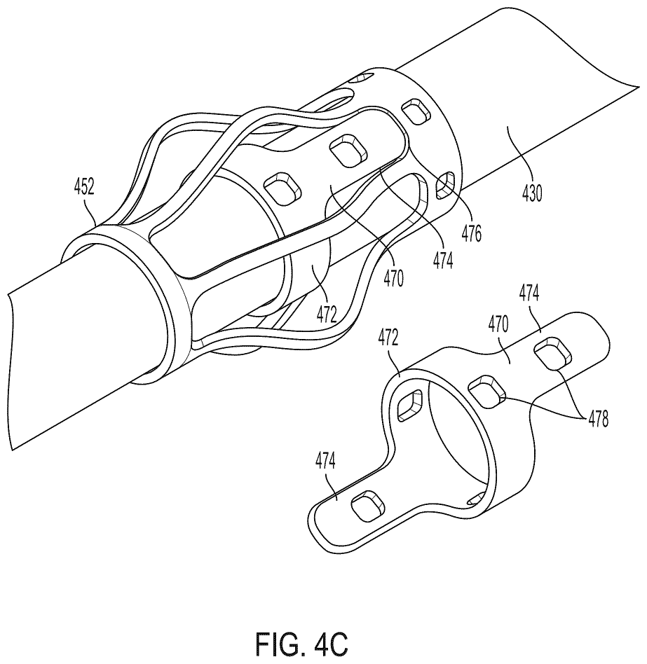

FIG. 4A illustrates a catheter 400 that includes another exemplary configuration of a spacer for spacing a shield from one or more shock wave emitters. Catheter 400 may be used for catheter 110 of FIG. 1 and may be configured similarly to catheter 200 of FIG. 2. Catheter 400 includes one or more spacers 452. The spacers 452 include annular rings 454 that fit around elongate member 430. The annular rings 454 may be fixed in place on the elongate member 430 via a friction fit and/or via adhesive. Alternatively, the annular rings 454 may be free to slide along the elongate member 430. Projections 456 extend between the annular rings 454 and project outwardly from the elongate member 430. The shield 416 is spaced from one or more emitter 412 by the projections 456.

The spacers 452 may be made from a cylindrical body that is laser cut to form the annular rings 454 and strips of material that extend between the annular rings 454. The strips of material may be forced outwardly into the shape of the projections 456 and heat set to maintain their projected shape. The projections 456 may be sufficiently flexible that they can be compressed into a flatter configuration to facilitate a collapsed state of the enclosure 410. In other words, the spacers 452 may be compressed via a radially inward force that causes the projections 456 to flatten out. One or both of the annular rings 454 may be free to slide along the elongate member 430 to enable the projections 456 to flatten out. For example, a first annular ring 454-A may be attached to the elongate member 430 to maintain the position of the spacer 452 and a second annular ring 454-B may be free to move relative to the elongate member 430 such that the spacer 452 can lengthen and the projections 456 can flatten out. The ability of one or both of the annular rings 454 to move longitudinally allows for the spacer 452 to compress (reduce in diameter), which can facilitate collapse and/or folding of a surrounding enclosure and/or shield. The ability of the enclosure and/or shield to collapse and/or fold (and the spacers to compress) enables the catheter to have a lower profile, which may be useful for delivering the catheter to a treatment site or removing the catheter from a treatment site.

In examples in which the annular rings 454 are free to slide along the elongate member 430 to enable the projections 456 to flatten out as needed, a retainer may be used to keep the spacer 452 in position relative to the elongate member 430. FIG. 4B illustrates an exemplary retainer 460 that is configured to maintain a position of the spacer 452 along the elongate member 430. Retainer 460 may be a strip of material that is sized to fit between adjacent projections 456-A and 456-B and to extend from adjacent to the first annular ring 454-A to adjacent the second annular ring 454-B. Retainer 460 may be attached to the elongate member 430. The spacer 452 is not attached to the elongate member 430, but retainer 460 prevents the spacer 452 from moving longitudinally relative to the elongate member 430 beyond a minimal range. For example, if the spacer 452 is urged in a given longitudinal direction, the spacer 452 will move along the elongate member 430 in a longitudinal direction but only until one of the annular rings 454-A, 454-B abuts the retainer 460. The retainer 460 may be sized to provide little to no clearance between the ends of the retainer 460 and the annular rings 454-A, 454-B to minimize longitudinal movement of the spacer 452. The annular rings 454-A, 454-B are free to move further apart from one another, thereby enabling the projections 456 to flatten out as needed, such as to facilitate collapse of a surrounding enclosure and/or shield.

The retainer 460 and spacer 452 may be configured such that the retainer 460 maintains the circumferential location of the spacer 452 relative to the elongate member 430. For example, the ends of the retainer 460 may fit within slot portions 462 of the spacers 452 where the projections 456 meet the annular rings 454-A, 454-B. The interface between the retainer 460 and the slot portions 462 prevents the spacer 452 from rotating about the elongate member 430.

The example illustrated in FIG. 4B includes multiple retainers 460 for retainer the spacer 452, positioned around the circumference of the elongate member 430. In other examples, just a single retainer is provided for each spacer 452. The retainer 460 may include one or more holes 464 for accommodating adhesive that bonds the retainer 460 to the elongate member 430.

FIG. 4C illustrates an alternative configuration of a retainer for retaining a spacer on an elongate member. The retainer 470 of FIG. 4C includes a central annular portion 472 and two longitudinally extending extensions 474. Each longitudinally extending extension 474 fits within a slot portion 476 of the spacer 452 and prevent the spacer 452 from translating in a longitudinal direction along elongate member 430. The longitudinally extending extensions 474 provide for structure to hold the spacer in place on opposing sides of elongate member 430. The central annular portion 472 and/or the extensions 474 may be attached to the elongate member 430, such as by adhesive. The central annular portion 472 and/or the extensions 474 may include holes 478 for accommodating adhesive.

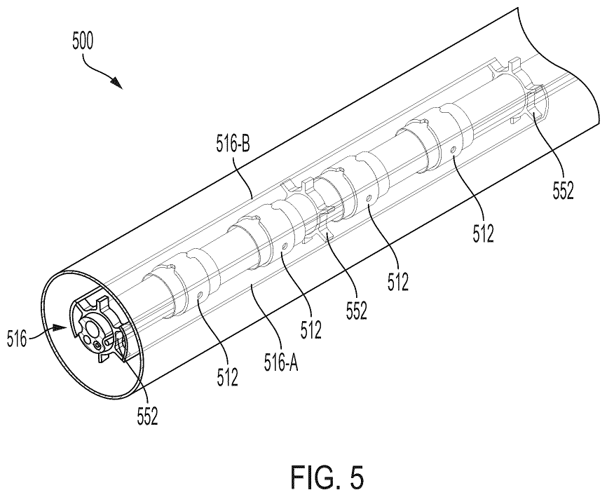

FIG. 5 illustrates a catheter 500 that includes shield strips 516 that are positioned in front of the shock wave emitters 512. Catheter 500 may be used for catheter 110 of FIG. 1 and may be configured similarly to any of the catheters discussed herein. Catheter 500 may include a spacer 552 that may be similar to spacer 352 of catheter 300 of FIGS. 3A and 3B. The implementation of FIG. 5 provides that the shield strips 516 are positioned directly in front of where shock waves are generated by shock wave emitters, without additional material surrounding or encircling other regions of the elongate member. The shield strips 516 may be attached to the spacers 352 such that the shield strips 516 are held in place. In the illustrated example, a first shield strip 516-A is positioned in front of a first set of shock wave emitters 512 and a second shield strip 516-B is positioned diametrically opposite to the first shield strip 516-B in front of a second set of shock wave emitters (not shown) that are diametrically opposite to the first set of shock wave emitters 512. The shield strips 516 may be advantageous in allowing for increasing fluid flow around the shock wave emitters 512, channeling a conductive fluid toward or alongside the emitters 512. The shield strips 516 may be advantageous in allowing for increased flexibility as compared to a tubular shield.

Any of the shields described herein, including shield 116 of FIGS. 1A and 1B, shield 216 of FIGS. 2A and 2B, shield 316 of FIGS. 3A and 3B, shield 416 of FIGS. 4A-4C, and shield strips 516 of FIG. 5, may include a polymeric material. The polymeric material may be a thermoplastic polyurethane, a polyether block amide, or another thermoplastic elastomer (e.g., polypropylene, polyethylene, polyvinyl chloride, polystyrene, polyethylene terephthalate, polyphenyl sulfone, or polycarbonate). The polymeric material may be the same as, or different from, the polymeric material of the enclosure that surrounds the shield.

In some implementations, the shield material can further include a radiopaque material so that the shield is visible under fluoroscopy. For example, small molecular weight salts, such as iodine-based or bromine-based monomers in conjunction with lithium and/or sodium, are examples of radiopaque materials that can be incorporated into a polymer material without compromising the structural characteristics of the polymer. Examples of metallic radiopaque materials that can be incorporated into a polymer material without compromising the structural characteristics of the polymer include, but are not limited to, compounds, powders, or nanoparticles containing iodine, barium, tungsten, tantalum, bismuth, or gold, or sulfates, oxides, trioxides, carbonates, subcarbonates, or oxychlorides thereof.

In some examples, the polymeric material has a Shore A hardness less than 100. The polymeric material may have a Shore A hardness up to 90. The polymeric material may have a density less than 1.15 g/cm3. The properties of the polymeric material may be selected to allow transmission of energy from shock waves generated at the shock wave emitters. The transmitted energy may be sufficient to cause modification of target lesions. The polymeric material may be configured have a specific gravity such that the shield is acoustically transparent to shock waves traveling through fluid within an enclosure. For example, the polymeric material may have a specific gravity close to that of saline, such as 1.12-1.56. The polymeric material may have a specific gravity that is greater than 1.0, such as greater than 1.1 or greater than 1.2. The polymeric material may have a specific gravity that is less than 2.0, such as less than 1.8, less than 1.6, or less than 1.4. The polymeric material may have a specific gravity that is in a range of 1.0 to 1.6. A thickness of the polymeric material may be in a range of 0.002 inches to 0.025 inches. The polymeric material may be sufficiently flexible that the shield can fold and/or compress.

FIG. 6 illustrates an example of a computing system 600 that can be used for one or more components of the system 100 of FIG. 1, such as energy source 120 and/or fluid source 145. System 800 can be a computer connected to a network, such as one or more networks of hospital, including a local area network within a room of a medical facility and a network linking different portions of the medical facility, or a wide-area network accessed through the internet or other means. System 600 can be a client or a server. System 600 can be any suitable type of processor-based system, such as a personal computer, workstation, server, handheld computing device (portable electronic device), such as a phone or tablet, or dedicated device. System 600 can include, for example, one or more of input device 620, output device 630, one or more processors 610, storage 640, sensor device 670, and communication device 660. Input device 620 and output device 630 can either be connectable or integrated with the computer.

Input device 620 can be any suitable device that provides input, such as a touch screen, keyboard or keypad, a button, a switch, a mouse, a gesture recognition component of a virtual/augmented reality system, or a voice-recognition device. Output device 630 can be or include any suitable device that provides output, such as a display, touch screen, haptics device, virtual/augmented reality display, indicator light, or speaker.

Storage 640 can be any suitable device that provides storage, such as an electrical, magnetic, or optical memory including a RAM, cache, hard drive, removable storage disk, or other non-transitory computer-readable medium. Communication device 660 can include any suitable device capable of transmitting and receiving signals over a network, such as a network interface chip or device. The components of the computing system 600 can be connected in any suitable manner, such as via a physical bus or wirelessly.

Processor(s) 610 can be any suitable processor or combination of processors, including any of, or any combination of, a central processing unit (CPU), field programmable gate array (FPGA), and application-specific integrated circuit (ASIC). Software 650, which can be stored in storage 640 and executed by one or more processors 610, can include, for example, the programming that embodies the functionality or portions of the functionality of the present disclosure (e.g., as embodied in the devices as described above), such as programming for performing energy delivery to one or more shock wave emitters for generating shock waves.

Software 650 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 640, that can contain or store programming for use by or in connection with an instruction execution system, apparatus, or device.

Software 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer-readable medium can include, but is not limited to, an electronic, magnetic, optical, electromagnetic, or infrared wired or wireless propagation medium.

System 600 may include a sensor device 670 that provides sensor data for processing by processor 610. Sensor device 670, in some embodiments, may be an imaging sensor that provides imaging data for a lesion being treated. In some embodiments, sensor device 670 may be a voltage sensor, a current sensor, a pressure sensor, a temperature sensor, or an optical sensor for providing data about a state of the catheter or a lesion.

System 600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 600 can implement any operating system suitable for operating on the network. Software 650 can be written in any suitable programming language, such as C, C++, Java, or Python. In various examples, application software embodying the functionality of the present disclosure can be deployed in different configurations, such as in a client/server arrangement or through a Web browser as a Web-based application or Web service.

System 600 may be configured to selectively control the delivery of energy from one or more of energy sources (e.g., a voltage pulse generator or a light energy source) to one or more acoustic energy emitters (e.g., a forward-firing emitter, a radially-firing emitter, an unenclosed emitter, or an enclosed emitter) depending on input from input device 620.

System 600 may be configured to tune the energy properties of energy delivered to one or more of the above-described emitters based on tissue properties received from sensor device 670. Tissue properties may include lesion tissue type (e.g., calcific, thrombic, fibrotic), lesion morphology (e.g., thickness, length, eccentricity).

The elements and features of the example catheters and catheter systems illustrated throughout this specification and drawings may be rearranged, recombined, and modified without departing from the present disclosure. For instance, the number, placement, and spacing of shock wave generating regions or emitters can be modified and the number, placement, and spacing of the enclosures of catheters can be modified without departing from the present disclosure.

Although the catheter devices described herein have been discussed primarily in the context of treating coronary occlusions, such as lesions in vasculature, the catheter devices described herein can be used for a variety of occlusions, such as occlusions in the peripheral vasculature (e.g., above-the-knee, below-the-knee, iliac, carotid, etc.). For further examples, various embodiments may be used for treating soft tissues, such as cancer and tumors (i.e., non-thermal ablation methods), blood clots, fibroids, cysts, organs, scar and fibrotic tissue removal, or other tissue destruction and removal treatments. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal, and destruction of tissue, or used in place of thermal treatments or cauterization for venous insufficiency and fallopian ligation (i.e., for permanent female contraception).

In one or more examples, the electrode assemblies and, catheters described herein could also be used for tissue engineering methods, for instance, for mechanical tissue decellularization to create a bioactive scaffold in which new cells (e.g., exogenous and endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

It will be understood that the foregoing is only illustrative, and that various modifications, alterations and combinations can be made by those skilled in the art without departing from the scope and spirit of the disclosure. Any of the variations of the various catheters disclosed herein can include features described by any other catheters or combination of catheters herein. Furthermore, any of the methods can be used with any of the catheters disclosed. Accordingly, it is not intended that the systems, catheters, and methods described herein be limited, except as by the appended claims.

1. A catheter for treating a lesion in a body lumen, the catheter comprising:
an elongate member;
an enclosure surrounding a distal region of the elongate member;
a shock wave emitter located within the enclosure;
a shield positioned between the shock wave emitter and the enclosure such that a shock wave emitted by the shock wave emitter passes through the shield before passing through the enclosure; and
a spacer that maintains a minimum spacing between the shock wave emitter and the shield.

2. The catheter of claim 1, wherein the shield is open at at least one end.

3. The catheter of claim 1, wherein the shield is free-floating within the enclosure.

4. (canceled)

5. The catheter of claim 1, wherein the spacer is configured to compress.

6. The catheter of claim 5, wherein at least a portion of the spacer is moveable relative to the elongate member to enable the spacer to compress.

7. The catheter of claim 6, comprising a retainer for retaining the spacer in position relative to the elongate member.

8. The catheter of claim 1, wherein the shield is tubular in shape.

9. The catheter of claim 1, wherein the shield is configured as a strip.

10. The catheter of claim 1, wherein the shock wave emitter comprises an electrode pair including a spark gap and the shield is located between the spark gap and the enclosure.

11. The catheter of claim 1, wherein the shield comprises a thermoplastic polyurethane or a polyether block amide.

12. The catheter of claim 1, wherein the shield comprises a material having a specific gravity of 1.0-1.6.

13. The catheter of claim 1, wherein the shield comprises a material having a Shore A hardness less than 100.

14. The catheter of claim 1, wherein the shield comprises a radiopaque material.

15-20. (canceled)
